(19) United States
(12) Patent Application Publication
(10) Pub. No.: US 2021/0100748 A1
(43) Pub. Date: Apr. 8, 2021

(54) Launch, Anti-radiation supplement

(71) Applicant: Tim Chrisman, (US)

(72) Inventor: Tim Chrisman, Falls Church, VA (US)

(73) Assignee: Tim Chrisman, Falls Church, VA (US)

(21) Appl. No.: 17/122,278

(22) Filed: Dec. 15, 2020

(51) Int. Cl. A61K 9/48; A61K 9/20; A61K 36/258; A61K 36/16; A61K 36/81; A61K 36/68; A61K 31/05; A23L 33/105; A23L 29/00

(52) U.S. Cl. CPC A61K 9/48; A61K 9/20; A61K 36/258; A61K 36/16; A23L 29/035; A61K 36/68; A61K 31/05; A23L 33/105; A61K 36/81

(57) ABSTRACT

Launch is a nutritional supplement designed to slow the negative effects of radiation on the body while an individual is traveling in the upper atmosphere or outer space. It combines 5 natural compounds shown in clinical trials to reduce radiation-induced inflammation, into a single supplement.

### FORMULATION

Ingredients are combined into two tablets

| | |
|---|---|
| 1,000 mg | Ginseng |
| | Standardized at 7% Ginsenosides |
| 500 mg | Ashwagandha (also known as Withania somnifera) |
| 500 mg | Resveratrol |
| 300 mg | Baccopa monnieri (also known as water hyssop) |
| 200 mg | Ginkgo Bilboa |

Supplement is to be take two times daily preferably with a meal to help increase the body's resistance to radiation

1. A tablet or capsule consisting essentially of not less than 450 mg and not more than 550 mg Ginseng Root standardized at 7% Ginsenosides, not less than 225 mg and not more than 275 mg Ashwagandha (also known as Withania somnifera), not less than 225 mg and not more than 275 mg Resveratrol, not less than 125 mg and not more than 175 mg Baccopa monnieri (also known as water hyssop) and not less than 75 mg and not more than 125 mg Ginkgo biloba.
